(19) United States
(12) Patent Application Publication
(10) Pub. No.: US 2004/0057869 A1
(43) Pub. Date: Mar. 25, 2004

(54) Gas exchange

(76) Inventor: John Dingley, Swansea (GB)

Correspondence Address: CONLEY ROSE, P.C., P. O. BOX 3267, HOUSTON, TX, 77253-3267, US

(21) Appl. No.: 10/432,884

(22) PCT Filed: Nov. 28, 2001
(86) PCT No.: PCT/GB01/05288

(30) Foreign Application Priority Data
Nov. 28, 2000 (GB) 0028987.6
Sep. 21, 2001 (GB) 0122757.8

(51) Int. Cl. A61M 1/14

(52) U.S. Cl. 422/48

(57) ABSTRACT

A method and apparatus (20) for maintaining a gas in a predetermined pressure range during a gas exchange process. The apparatus (20) includes a first conduit (21) having a gaspermeable membrane wall portion (25), at least one inlet port (26) for introducing a first gas into the apparatus, and a reservoir (28) arranged to contain the first gas. The method and apparatus are particularly suitable for oxigenating an extracorporeal flow of blood.

The present invention is concerned with a method of maintaining gas in a predetermined pressure range during a gas exchange process, and apparatus for performing a method of maintaining gas in a predetermined pressure range during a gas exchange process. The invention is particularly concerned with maintaining gas (such as oxygen) in a predetermined pressure range during the oxygenation of blood. The present invention is also concerned with the recirculation of a flow of gas around a conduit containing a membrane whilst maintaining the gas flowing across the membrane within a predetermined pressure range.

When cardiac surgery is performed, one common technique used is to stop the heart and use a mechanical device to pump blood around the body of the unconscious patient which also adds oxygen and removes carbon dioxide from the blood of the patient. The machine used to carry out this procedure is known as a cardiopulmonary bypass machine. Once the surgery is complete, the patient is removed from the cardiopulmonary bypass machine and the normal function of the heart and lungs are restored. The part of the bypass machine that adds oxygen to the blood and removes waste carbon dioxide from it is called the oxygenator.

One common type of oxygenator in commercial use includes a gas permeable membrane. A gas mixture containing oxygen (typically a mixture of nitrogen and oxygen) is passed along one face of a membrane whilst the blood of the patient is passed along the opposite face of the membrane. Oxygen diffuses through the membrane into the blood and waste carbon dioxide diffuses from the blood through the membrane into the gas stream. The carbon dioxide is then carried away in the gas stream and exhausted to atmosphere.

The system described above is adequate for normal use but is wasteful of fresh gases as the gas stream is vented to the atmosphere. Alternative gases to oxygen/nitrogen mixes in the gas stream may be desirable in certain circumstances. Such alternatives may, for example, include more expensive gases, such as the gas xenon which is advantageous for its anaesthetic and/or brain protecting properties. The use of such expensive gases has previously been restricted due to the economic disadvantages when they are exhausted to the atmosphere.

It is therefore an aim of the present invention to alleviate the problems of the prior art highlighted above.

Therefore, according to the first aspect of the present invention, there is provided a method of maintaining a gas in a predetermined pressure range during a gas exchange process which includes:

circulating a gas in a first conduit having a gas-permeable membrane wall portion;

permitting the gas to diffuse through the wall portion into a second conduit;

replenishing the diffused gas via at least one inlet port; permitting the gas to transfer from the first conduit to a gas-containing reservoir if the gas pressure exceeds the predetermined pressure range or the gas exceeds a predetermined volume, and permitting the gas to be transferred from the gas-containing reservoir to the first conduit if the pressure in the first conduit falls below the predetermined pressure range or the volume of the gas falls below the predetermined volume, so as to maintain the pressure of the first gas in the first conduit substantially within the predetermined pressure range.

It is particularly preferred that the predetermined pressure range includes ambient pressure. Desirably, the first circulating conduit has a physical volume which is substantially the same as the predetermined volume.

The use of the reservoir in the method according to the invention can allow small imbalances to occur between gas uptake and delivery in the first conduit, substantially without fresh gases being lost to the atmosphere. If a large accidental excess of fresh gas were to be delivered to the first conduit, the excess gas would move into or even emerge from the end of the reservoir, and there would be no dangerous pressure build up.

The second conduit typically contains an extracorporeal flow of blood. When the second conduit contains blood, it is preferred that the gas in the first conduit includes oxygen. The gas may optionally include a gas suitable for use as an anaesthetic, such as, for example, xenon, or another gas in Group VIII of the Periodic Table of the Elements (such as krypton). Alternatively, the gas may optionally include any suitable gas for use as a brain protecting drug. It is envisaged that the anaesthetic gas and the gas for use as a brain protecting drug may be the same.

The membrane wall portion is preferably an oxygenator membrane. Such a membrane should be substantially inert to reactions with blood, and should be impermeable thereto. Preferably the membrane wall portion is of a gas-permeable film of a polymer such as microporous polypropylene hollow fibres, or alternatively a silicone rubber membrane. However, it is envisaged that any commercial oxygenator membrane may be utilized.

The gas-permeable membrane wall portion is arranged to permit the gas, typically a mixture containing oxygen, to diffuse through the membrane from the first conduit to the second conduit, and a second gas to diffuse through the membrane from the second conduit to the first conduit. The second gas typically includes carbon dioxide. It is therefore preferable to include a further step whereby the carbon dioxide is removed from the gas contained within the first conduit.

The membrane of the oxygenator, through which gas exchange takes place, is preferably substantially at atmospheric pressure on the gas side. If the mean gas pressure is too high, gas bubbles may undesirably be forced through the membrane to the blood flow. It is therefore envisaged that the internal surface of the first conduit has a low resistance to flow (typically by having a sufficiently large diameter). In a particularly preferred embodiment, the pressure may be maintained substantially at atmospheric pressure by positioning the reservoir substantially adjacent the gas permeable membrane.

It is preferred that the gas is circulated around the first conduit by a motorised pump, such as an oscillating diaphragm pump or a small turbine type pump.

The reservoir may be an open ended conduit, vented to, for example, ambient atmosphere, or alternatively, a vessel of variable volume, such as an inflatable bellows, bag or the like, manufactured from suitable gas-impermeable flexible sheeting. Preferably, when the reservoir is a vessel of variable volume, the gas is added to the first conduit so as to avoid overfilling or complete emptying the vessel of the gas.

It is preferred that the gas should include a mixture of at least two components. Preferably, each component of the gas is provided with an individual inlet port therefor. However, it is envisaged that each component of the gas mixture may enter through the same port.

The gas typically includes oxygen and xenon. It is desirable that oxygen is present in an amount of from about 0 to 100%, preferably 30 to 100% (further preferably 30% to 80%). Desirably, xenon is present in an amount of from about 0% to 100% (preferably 0% to 79%,further preferably 20 to 70% when the Xenon is used as an anaesthetic or for its neuro protection properties).

According to a first embodiment of the present invention, each inlet port is in communication with the first conduit.

Advantageously, each component of the gas is introduced by controlled injection. The control of the injection may be manual or automatic. The flow of gases may be continuous or intermittent.

According to a second embodiment of the present invention, a first inlet port is in communication with the reservoir and a second inlet port is in communication with the first conduit. Typically, the first inlet port introduces oxygen. Preferably the second inlet port introduces xenon. In this embodiment, it is preferred that the flow of oxygen through the first inlet port should be continuous.

Preferably the flow of xenon through the second inlet is by controlled injection; the controlled injection may be a continuous or intermittent process.

The second embodiment has the advantage that if no fresh gas is manually or automatically added (due to malfunction for example), oxygen will then be slowly drawn into the first conduit from the reservoir as gas is absorbed into the blood across the oxygenator membrane, so as to assist in maintaining a patient's vital functions.

If too much gas such as xenon were to be accidentally introduced into the first conduit, then any excess should be flushed away by the continuous oxygen flow. Advantageously, the reservoir is mainly filled with oxygen at all times, even if a large accidental bolus of xenon is given. This is desirable in terms of safety for the situation described in the second embodiment of the invention to occur efficiently. Accidental large xenon boluses could otherwise fill the safety gas reservoir mainly with xenon rather than oxygen, which is, of course, undesirable.

According to a particularly preferred embodiment of the present invention, there is provided a method of oxygenating blood, which method includes:

circulating oxygen in a first conduit having a gas permeable membrane wall portion;

permitting the oxygen to diffuse through the wall portion into a second conduit;

replenishing the diffused oxygen via at least one inlet port in the first conduit;

permitting the oxygen to transfer from the first conduit to an oxygen-containing reservoir if the gas pressure exceeds the predetermined pressure range or the gas exceeds a predetermined volume, and permitting the gas to be transferred from the oxygen-containing reservoir to the first conduit if the pressure in the first conduit falls below the predetermined pressure range or the volume of the gas falls below the predetermined volume, so as to maintain the pressure of the oxygen in the first conduit substantially within a predetermined pressure range.

The blood is preferably an extracorporeal flow of blood.

The method is preferably substantially as described hereinbefore.

The method according to the present invention is particularly advantageous as it permits exchange of gases to occur in an extracorporeal flow of blood, within economy of use of fresh gases.

According to a second aspect of the present invention, there is provided apparatus for maintaining gas in a predetermined pressure range during a gas exchange process which apparatus includes:

a first conduit having a gas-permeable membrane wall portion;

at least one inlet port for introducing a first gas into the apparatus; and

a reservoir arranged to contain the first gas.

The apparatus may be used in the method of maintaining a gas in a predetermined pressure range during a gas exchange process substantially as described hereinbefore. The apparatus advantageously substantially mountains gas flowing across the membrane wall portion within a predetermined pressure range.

The reservoir may be an open-ended conduit, vented to, for example, the ambient atmosphere, or alternatively, a vessel of variable volume, such as an inflatable bellows, bag or the like, manufactured from suitable gas-impermeable flexible sheeting.

It is envisaged that when the system includes a vessel of variable volume to act as a reservoir the system optionally includes a control port arranged to permit gas to exit the apparatus if the pressure in the system exceeds ambient pressure i.e. the inflatable bellows is full and permits entry of I) the first gas; ii) one of its component gases or iii) ambient air, if the pressure in the system falls below ambient (i.e. the inflatable bellows, bag or the like. become substantially empty).

Advantageously, when the apparatus is used for the oxygenation of blood, the apparatus includes means for removing carbon dioxide from, for example, the first conduit.

The apparatus is typically maintained substantially at atmospheric pressure, in particular about the membrane wall portion. It is envisaged that the first conduit may have a diameter sufficiently large that provides a low resistance to the flow of gas. In a preferred embodiment the reservoir is typically positioned substantially adjacent the gas-permeable membrane wall portion.

Typically, the gas-permeable membrane wall portion is an oxygenator membrane, substantially as described above.

The apparatus typically includes a first inlet port (preferably for the introduction of oxygen) and a second inlet port (preferably for the introduction of a second gas such as xenon).

According to a first embodiment of the second aspect of the present invention, the first inlet port and the second inlet port are in communication with the first conduit.

According to a second embodiment of the second aspect of the present invention, the first inlet port is in communication with the reservoir and the second inlet port is in communication with the first conduit.

Preferred features of the present invention will now be described, by way of illustration only, with reference to the accompanying Figures, in which:

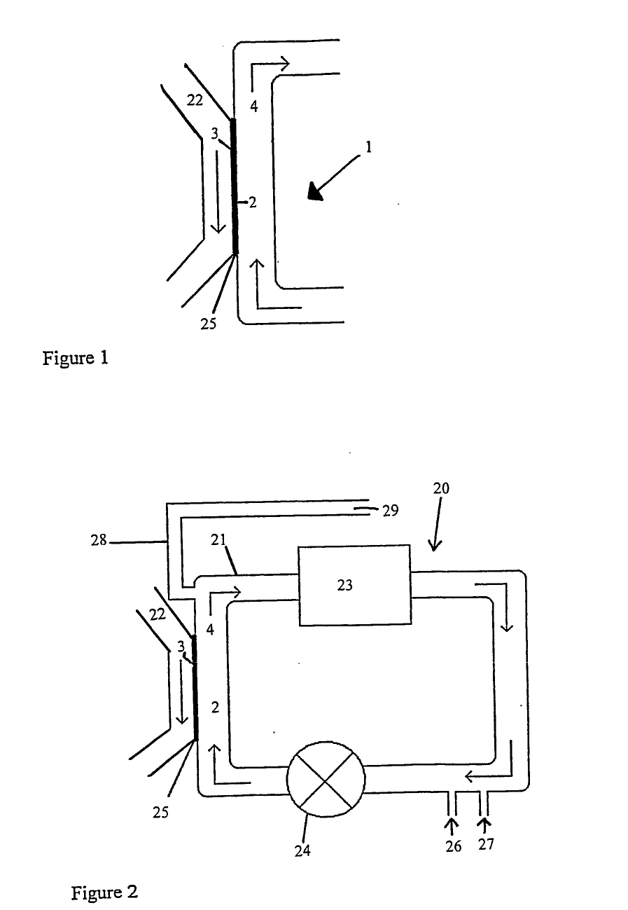

FIG. 1 represents prior art gas exchange apparatus;

FIG. 2 represents apparatus according to a first embodiment of the present invention;

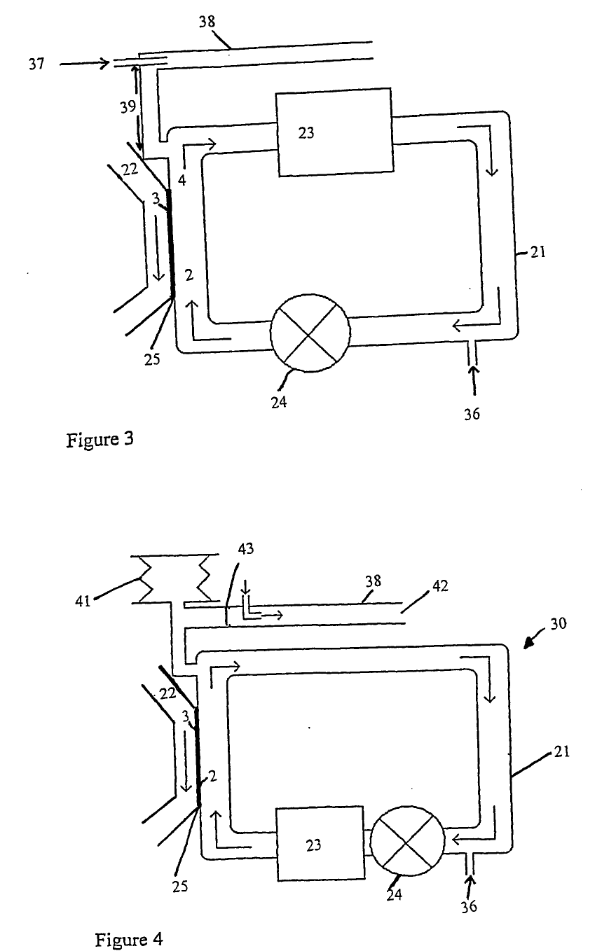

FIG. 3 represents apparatus according to a second embodiment of the present invention; and

FIG. 4 represents apparatus according to a further embodiment of the present invention.

Referring to FIG. 1, there is shown a known type of oxygenator indicated by the numeral 1. A gas mixture containing oxygen 4 (usually a mixture of nitrogen and oxygen) is passed along one face of the membrane 2, and the blood of the patient is pumped along the opposite face of the membrane 3. Oxygen diffuses through the membrane into the blood and waste carbon dioxide diffuses from the blood through the membrane into the gas mixture 4. The carbon dioxide is then carried away in the gas stream 4 and vented to ambient atmosphere.

Referring to FIG. 2, where like numerals have been used to indicate like parts to those shown in FIG. 1, there is illustrated apparatus according to the first aspect of the present invention indicated by the numeral 20.

The gases passing along the gas face 2 of the oxygenator membrane 25 are recirculated around a loop of hollow tubing 21. The blood 22 of the patient passes along the other side of the oxygenator membrane 25 in conventional manner. At the membrane 25, waste carbon dioxide diffuses from the blood 22 to the gas side of the membrane 2, into the gas stream 4. This waste carbon dioxide is removed from the gas stream 4 by passing gas stream 4 through a container filled with carbon dioxide scrubbing material 23. The gases are recirculated around the loop of tubing 21 by a motorised pump 24. At the oxygenator membrane 25, oxygen diffuses from the gas stream 4 through the membrane 24 into the blood 22 of the patient.

As the carbon dioxide is being removed, the volume of gas in the loop of tubing 21 slowly falls with time as gas (mainly oxygen) moves from the gas pathway 4 into the blood stream 22 across the membrane 25. The rate at which this occurs would typically be about 250 ml per minute. Fresh oxygen is added to the gas loop 21 through port 26 and xenon through port 27. The concentration of each constituent gas within the gas loop is monitored in order to guide this gas addition process.

As a balance is occurring between gas uptake into the blood and fresh gas delivered to the gas loop, the pressure in the gas loop is kept under control. This is usually at or near atmospheric pressure. This is achieved using an open ended reservoir 28 connected to the gas loop 21 which allows small imbalances to temporarily occur between the rate of gas uptake and fresh gas addition to the loop, without excessive pressure buildup. If slightly too much fresh gas is temporarily delivered through ports 26 and 27, some of the excess gas can emerge down the reservoir 28 temporarily without being vented to atmosphere from its distal open end 29. After further gas uptake through the membrane 25 takes place, the gas which was forced into the reservoir 28 is drawn back into the loop 21 from the reservoir 28 as the gas volume within the loop 21 starts to fall once again.

Referring to FIG. 3, where like numerals have been used to indicate like parts to those shown in FIGS. 1 and 2, there is illustrated apparatus according to the second embodiment of the invention is indicated by the numeral 30.

An open-ended reservoir is provided 38. Into this runs a constant flow of oxygen through inlet port 37. Xenon is delivered in small quantities as required to the gas loop 21 through inlet port 36. If xenon is transiently delivered to the gas loop through inlet port 36 at a rate faster than the total rate of gas uptake from the loop 21 into the blood 22, the excess gas volume will move up the reservoir 38 as described in FIG. 2 above. If this “excess volume” 39 exceeds the volume of reservoir tube between the loop 21 and the oxygen inlet port 37, then any more excess gas will be flushed out of the reservoir 38 by the oxygen flow through inlet port 37.

Xenon can be added in boluses to the loop 21 with pauses to measure the new gas composition within the loop 21, and this allows the operator (manual or automatic) to keep the percentages of each gas component within the mixture substantially constant in the loop.

The system described in FIG. 1 is regarded as an “open” system, which means that no fresh gas passes through the system and therefore through the oxygenator, more than once.

The text relating to FIGS. 2 and 3 describes these systems being used “fully closed”, as this is the most economical and most desirable mode of operation. This means that fresh gases are allowed to enter the loop at a rate more or less equal to the uptake of each of these gases into the blood via the oxygenator. This is the most efficient mode of operation in terms of gas consumption, and therefore running costs.

The system (comprising; oxygenator, gas recirculation pump, carbon dioxide absorber plus mechanism allowing this “loop” to be open to atmosphere such as a reservoir limb) can also be used as: “semi-closed”. In this embodiment, fresh gases (for example oxygen and xenon) are introduced to the loop through a port or ports, for example ports 26 and 27 in FIG. 2. The flow of these gases is arranged to be continuous and the flow of each gas into the loop is arranged to slightly exceed the uptake rate of each gas from the loop by the blood via the oxygenator membrane. In this mode, there is a continuous “spill” of excess gas from the system which allows the loop to be functionally open to atmosphere (such as the reservoir limb in FIG. 2). At the same time, fresh gases partially recirculate around the loop a few times before exiting the system. This mode of operation uses less fresh gas than the open system described in FIG. 1, as the fresh gas is partially recirculated. It uses more fresh gas than the fully-closed modes of operation described earlier in FIGS. 2 and 3 as in fully-closed mode, the fresh gases are fully recirculated until taken up into the blood. Semi-closed operation has an advantage however, as when in use the gas composition in the loop reaches an equilibrium and therefore stays relatively constant. This means that though less economical than the fully-closed modes of use described in FIGS. 2 and 3, it does not require such a high level of vigilance in terms of monitoring and control as is necessary with the fully-closed modes of operation, in order to be used safely.

Referring to FIG. 4, oxygen plus or minus xenon is taken up from loop across oxygenator membrane 2 by blood of patient, the volume of gas in loop 21 and bellows 41 therefore decreases. The bellows 41 does not collapse under its own weight and eject its contents out of end of reservoir limb 42, because there is a one way valve 43 in the reservoir limb which only lets gas move INTO the loop and not out of the loop.

When the bellows 41 empties, the continued gas consumption from the loop across the oxygenator is replaced by oxygen drawn into the loop from the reservoir limb at the same rate. This gas is drawn into the loop 21 via the aforementioned passive one way valve which requires a very small pressure difference across it in order to open.

If xenon is injected into the loop 21 via the port 36, the bellows 41 will fill to accommodate the extra added gas.

It will not leak from the reservoir limb as the one way valve 43 closes.

Therefore the gas side of the oxygenator is protected from negative pressure build up by the fact that extra oxygen would be drawn into the loop 21, and protected from positive pressure build up by the fact that the height of the bellows 41 would increase if extra gas were added to the loop. If the operator does nothing, oxygen is always added to the loop automatically as fast as gas is taken out via the oxygenator 2. The bellows 41 allows added gas to be accommodated without pressure build up. The bellows 41 and valve 43 are positioned substantially adjacent the gas exit side of the oxygenator 2 to keep the pressure in the apparatus low as substantially at atmospheric pressure.

The system described in FIGS. 3 and 4 is particularly desirable as when the gas mixture in the first conduit (21) comprises a mixture of oxygen and another gas such as xenon, then the volume of gas taken up across the membrane from the conduit equals the oxygen uptake per minute plus the xenon uptake per minute. If no fresh xenon is added, this combined volume loss is replaced with oxygen drawn into the first conduit (21) from the oxygen filled reservoir system. Therefore, in the absence of xenon addition to the loop or first conduit (21), the oxygen concentrations in the first conduit (21) will slowly rise. It is envisaged that in use, this slow rising oxygen concentration is counterbalanced by small repeated xenon injections into the loop (21). The end result is a substantially constant xenon and oxygen concentrations within the loop (21). The system therefore has inherent safety, as failure to inject xenon causes the oxygen concentrations in the loop (21) to slowly rise which is important to sustain life.

1. Apparatus for maintaining gas in a predetermined pressure range during a gas exchange process which apparatus includes:
a first conduit having a gas-permeable membrane wall portion;
a reservoir arranged to contain the first gas; the reservoir being in communication with the first conduit; and
a first inlet port for introducing a first component of the first gas into the reservoir and a second inlet for introducing a second component of the first gas into the first conduit.

2. Apparatus according to claim 1, wherein the reservoir is substantially adjacent the gas permeable membrane.

3. Apparatus according to claim 1 or 2, wherein the first conduit is a continuously circulating conduit.

4. Apparatus according to any preceding claim, wherein the reservoir is an open ended conduit.

5. Apparatus according to any preceding claim, wherein the reservoir is a vessel of variable volume.

6. Apparatus according to claim 5, wherein the vessel of variable volume is an inflatable bellows, bag or the like.

7. Apparatus according to claim 5 or 6, wherein the vessel of variable volume is manufactured from gas impermeable sheeting.

8. Apparatus according to any of claims 5 to 7, which includes a control port preferably arranged to permit gas to exit the apparatus if the pressure in the system exceeds ambient pressure.

9. Apparatus according to any preceding claim, wherein the membrane wall portion is an oxygenator membrane.

10. Apparatus according to any preceding claim, wherein the membrane wall portion is substantially inert to reactions with blood and impermeable to blood.

11. Apparatus according to any preceding claim, wherein the membrane wall portion is of a gas-permeable film of a polymer such as microporous polyprolylene hollow fibres, or a silicone rubber membrane.

12. Apparatus according to any preceding claim, wherein the gas-permeable membrane wall portion is arranged to permit the gas to diffuse through the membrane from the first conduit to a second conduit, and a second gas to diffuse through the membrane from the second conduit to the first conduit.

13. Apparatus according to any preceding claim, which includes a carbon dioxide removal means.

14. Apparatus according to any preceding claim, wherein the first conduit has an internal surface of low resistance to flow.

15. Apparatus according to any preceding claim, wherein the first inlet port introduces oxygen and the second inlet port introduces xenon.

16. Apparatus according to any preceding claim, wherein the apparatus includes means for removing carbon dioxide from the first conduit when the apparatus is used for the oxygenation of blood.

17. Apparatus for maintaining gas in a predetermined pressure range during a gas exchange process which apparatus includes:
a first circulating conduit having a gas permeable membrane wall portion;
at least one inlet port for introducing a first gas into the apparatus; and
a reservoir arranged to contain the first gas, the reservoir being in communication with the circulating conduit, wherein the conduit is arranged such that substantially all of the first gas circulating in the first conduit exit the conduit via the gas membrane.

18. Apparatus according to claim 17, which includes a first inlet port for introducing a first component of the first gas into the apparatus and a second inlet for introducing a second component of the first gas into the first conduit.

19. Apparatus according to claim 17 or 18, wherein the first circulating conduit is a closed loop.

20. Apparatus for maintaining gas in a predetermined pressure range during a gas exchange process which apparatus includes:
first conduit having a gas permeable membrane wall portion;
a reservoir arranged to contain the first gas, the reservoir being in communication with the first conduit; and being substantially adjacent the gas membrane wall portion; and
at least one inlet port got introducing a first gas into the apparatus.
